(19) United States
(12) Patent Application Publication
(10) Pub. No.: US 2002/0151940 A1
(43) Pub. Date: Oct. 17, 2002

(54) Transcutaneous spine trauma and disorders treatment using ultrasonically induced confined heat (ulich) zone

(75) Inventors: Yoseph Bar-Cohen, Seal Beach, CA (US); J. M. Kabo, Granada Hills, CA (US); Helenann Kwong-Fu, La Canada Flintridge, CA (US); Paul L. Lee, San Marino, CA (US); Jeffrey C. Wang, Los Angeles, CA (US); Ajit K. Mal, Santa Monica, CA (US)

Correspondence Address: FULBRIGHT & JAWORSKI L.L.P., Robert Berliner, Twenty-Ninth Floor, 865 South Figueroa, Los Angeles, CA, 90017-2571, US

(73) Assignee: University of California

(21) Appl. No.: 10/093,866

(22) Filed: Mar. 7, 2002

Related U.S. Application Data
(60) Provisional application No. 60/274,111, filed on Mar. 8, 2001.

(51) Int. Cl. A61F 7/00

(52) U.S. Cl. 607/99

(57) ABSTRACT

A method of heating a tissue using pulsed focused ultrasonic waves, and an apparatus for carrying out the method. The method comprises depositing an amount of ultrasound energy sufficient to heat the selected site without adversely damaging other parts of the tissue. The apparatus comprises an ultrasonic source for producing pulsed ultrasonic waves, a lens for focusing the ultrasonic waves, and a controller for controlling wave frequency and transmission parameters of the ultrasonic waves. The method and apparatus can be used to treat spine or other joint disorders associated with lax ligamentous tissues, i.e., soft tissue disruption.

### CROSS REFERENCE TO RELATED APPLICATIONS

This application is based on provisional application No. 60/274,111, filed on Mar. 8, 2001.

### FIELD OF INVENTION

This invention relates generally to medical treatment using ultrasonic waves.

### BACKGROUND OF THE INVENTION

The orthopedic medical community is seeking to eliminate the need for invasive surgical procedures in the treatment of spine trauma, preferably by performing the desired treatment procedure in an outpatient setting to substantially reduce costs and with minimal risks to the patient. Currently, existing methods of treatment require an extensive surgical incision, hospital stay, anesthesia, and prolonged recuperation time. The desired noninvasive capability must produce the same results as conventional surgery, while decreasing morbidity, complications, hospital costs, insurance and patient costs. Decreased recovery time after a noninvasive procedure will allow patients to return to work much more rapidly, resulting in decreased costs to society and an overall increase in productivity. Such a procedure could be used for clinical applications such as herniated intervertebral discs in the spine, arthritis in the spine, shoulder and knee instability and tumors in various locations in the musculoskeletal system. Treatments utilizing ultrasound hold much promise for the development of noninvasive surgical procedures.

Unger U.S. Pat. No. 6,088,613 discloses a method of magnetic resonance using focused surgical and therapeutic ultrasound involving the application of ultrasound to a region to carry out surgery. The method does not use ultrasound for non-surgical purposes and does not use pulsed focused ultrasonic waves. Watmough et al. U.S. Pat. No. 4,646,756 discloses an ultrasound unit producing ultrasound beams for the clinical application of hyperthermia to tumor cells in soft tissue. The beams are not pulsed beams. Lidgren et al. U.S. Pat. No. 6,254,553 discloses a device for non-invasive treatment of biological tissue, including intervertebral disc tissue, using ultrasound. The treatment does not involve the use of pulsed ultrasonic waves. Oppelt et al. U.S. Pat. No. 5,759,162 discloses an apparatus for treatment of a body tissue using ultrasound. The treatment does not involve the use of pulsed ultrasonic waves. Hutchinson et al. U.S. Pat. No. 6,135,971 discloses an apparatus for generating an ultrasound beam for deposition of ultrasound energy in body tissue. The beam is not a pulsed beam. Klopotek U.S. Pat. No. 6,113,559 discloses a method and apparatus for reducing skin wrinkles, including applying a focused ultrasound beam to a region of skin. The method and apparatus do not involve the use of pulsed ultrasonic waves.

Chapelon U.S. Pat. No. 5,743,863 discloses a high-energy ultrasound therapy method and apparatus producing ultrasound beams. The patent refers to “acoustic waves of a pulsed type” but indicates that work done with this type of acoustic wave cannot be assimilated into work at the basis of the Chapelon invention. Do-huu et al. U.S. Pat. No. 4,586,512 discloses a device for therapeutic heating of biological tissues using ultrasound. The patent suggests that pulse modulation could be used to regulate heat intensity, but does not further elaborate. Klopotek U.S. Pat. No. 6,325,769 discloses a method and apparatus of reducing skin wrinkles, including applying a focused ultrasound beam to a region of the skin. The patent refers to “[a]nother mechanism” using what it refers to as “pulsed acoustic waves” but does not further define what is meant.

Rolt et al. U.S. Pat. No. 5,501,655 discloses an ultrasound hyperthermia applicator suitable for medical hyperthermia treatment, and method for using the same. The patent describes the use of pulsed ultrasound signals produced by a signal generator driving an ultrasound transducer. However, the invention as described by Rolt et al. makes it apparent that there was no appreciation of the ability to use pulsation of an ultrasound beam to deposit at a selected site an amount of ultrasonic wave energy sufficient to heat the selected site without adversely damaging other areas of the body. Instead, the invention requires the use of at least two ultrasound transducers simultaneously directing focused ultrasound beams to a single site, i.e., using the known technique of concentrating energy applied from different directions.

The teachings of all of the above U.S. Patents are hereby incorporated by reference.

### SUMMARY OF THE INVENTION

The present invention relates to a novel method of using pulsed ultrasonic waves to induce a confined heated zone at a selected site in tissue of the body. In particular, this method is used to treat spine trauma and joint disorders in a non-invasive manner.

The method comprises the steps of directing pulsed ultrasonic waves to a selected site of a body tissue, then depositing at the site an amount of ultrasonic wave energy sufficient to heat the site without adversely damaging other areas of the body or tissue. Preferably, the waves are pulsed focused ultrasonic waves.

The present invention can induce a heated zone in a tissue without the need for a physically invasive operation or procedure. The method can be suitable for treatment of internal tissues, particularly for transcutaneous treatment of spine or other joint disorders associated with lax ligamentous tissues, i.e., soft tissue disruption. The method uses ultrasonically induced confined heat (ULICH) to rapidly perform a noninvasive treatment at low cost and minimum inconvenience, with rapid recovery, potentially minimum side effects and no inflammation or infection that may result from a surgical insertion of tools, devices or end effectors. The procedure can be administered in an outpatient setting, and without the need for anesthesia and its associated complications.

The present invention also provides an apparatus for heating a body tissue comprising an ultrasonic source for producing pulsed ultrasonic waves, a lens for focusing the pulsed ultrasonic waves on a selected tissue site, and a controller for controlling wave frequency and transmission parameters. The apparatus can heat a body tissue by depositing an amount of ultrasonic wave energy sufficient to heat the selected tissue site without adversely damaging other areas of the tissue.

The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

FIG. 1 is a schematic block diagram of an ultrasonic setup that is used to induce heat in muscle fibers;

FIG. 2 is a diagram of a transducer assembly that is used to induce a confined heated zone;

FIG. 3 is a graph showing temperature as a function of the frequency of a transducer; and

FIG. 4 is a schematic view of a transducer setup for the ULICH treatment.

### DETAILED DESCRIPTION OF THE INVENTION

The terms “ultrasonic” and “ultrasound” refer to an acoustic wave with a frequency from about 100 KHz to about 4 MHz.

The term “tissue” means a group of similarly specialized cells that perform a common function. Tissues compose the organs and other structures of living organisms.

The term “internal tissue” refers to any tissue that is inside the body but does not encompass tissue associated with skin or the three layers of the skin, namely the stratum corneum, the epidermis, and the dermis.

The term “confined heated zone” means a site of an internal tissue that is heated without causing measurable changes in regions of the tissue surrounding the site.

The term “transducer assembly” means a unit for producing ultrasonic waves, comprising an ultrasonic transducer such as a piezoelectric crystal and an external housing for encasing the transducer.

One object of the present invention is to provide a medical treatment that uses pulsed focused ultrasound to induce a confined heated zone in a tissue of the body. Another object of the present invention is to provide a medical treatment for spine trauma and other joint disorders that does not require incision or physical insertion of tools.

High power ultrasound generates heat as a result of induced cavitation phenomenon. Focusing the wave and using pulses instead of continuous signals allows for confining the level of input energy both in time and space, permitting effective control of the induced level of heat and the affected zone. This control capability is essential to minimizing risk associated with rising temperature and the area that is affected by the treatment.

The amount of heat deposited in a tissue by pulsed focused ultrasonic waves can be regulated by adjusting ultrasonic wave control parameters such as frequency, pulse duration, signal amplitude, spectral content, pulse rate and pulse shape. The frequency, which in this invention can be considered the spectral content, can range from about 100 KHz to about 4 MHz, preferably from about 500 KHz to about 900 KHz. Pulse duration can be from microseconds to seconds, preferably from about 10 microseconds to about 100 microseconds. Signal amplitude (prior to being amplified) can be in the range of about 1V to about 20V, preferably from about 10V to about 15V. The pulse rate can be from about 100 repetitions per second to about 1000 repetitions per second, preferably from about 100 repetitions per second to about 300 repetitions per second. Advantageous pulse shapes include square-wave and sine-wave shapes.

Thermal spikes induced by focused ultrasonic waves can be used to treat a localized area of soft tissues and disk elements in the vicinity of the spine and other internal body regions. Such local heating affects only the cross linking of the collagen causing the tissues to shrink and thus restoring stability to the spine, and/or reversing the weakness in the disk element resulting in a bulging disk. In an extreme case, it may also be possible to vaporize the nucleus pulposus to relieve the pressure exerted on the spinal cord without significant damage to the surrounding structures. Focused ultrasonic waves can induced tissue changes including tissue shrinkage, heating, or necrosis of pathological detrimental tissue to achieve improved clinical conditions.

The present invention provides a method of heating a tissue of the body of a life form, such as a human patient, comprising the steps of directing pulsed ultrasonic waves, preferably pulsed focused ultrasonic waves, to a selected site of the tissue, then depositing at the site an amount of ultrasonic wave energy sufficient to heat the site without adversely damaging other areas of the body or tissue.

Shape and dimensions of a heated site can be determined by scanning the heated site with a pulsed ultrasonic wave using a beam steering method.

Another aspect of the present invention is an apparatus for heating a tissue of the body of a life form, the apparatus comprising: (a) an ultrasonic source, preferably an ultrasonic transducer, for producing pulsed ultrasonic waves; (b) a lens, preferably an ultrasonic lens, for focusing the pulsed ultrasonic waves on a selected site of the tissue; and (c) a controller for controlling wave frequency and transmission parameters of the pulsed ultrasonic waves so that ultrasonic wave energy is deposited at the selected site in an amount sufficient to heat the selected site without adversely damaging other areas of the tissue. In a preferred embodiment, a single ultrasonic transducer comprises both the ultrasonic source and the lens.

Resonant Frequency of Focused Ultrasonic Waves

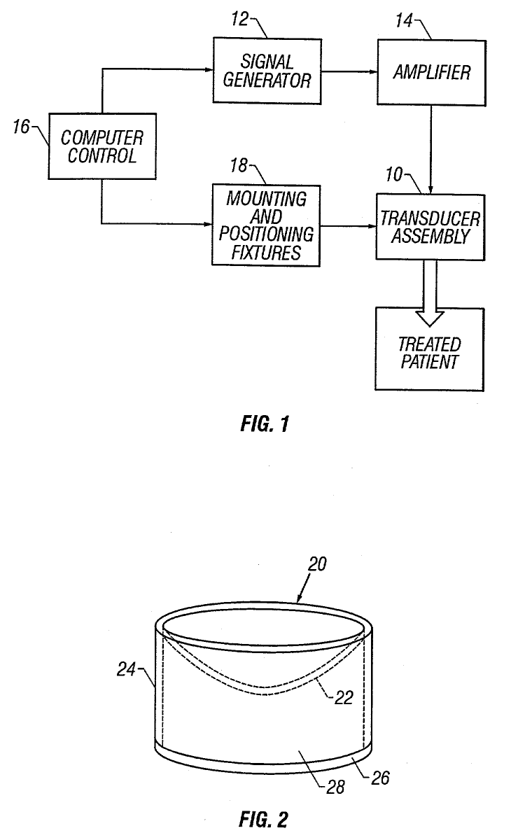

Focused ultrasonic waves were used in the frequency range of about 300 to about 650-KHz to induce confined heat. Test results showed that at about 529-KHz a resonance behavior occurred and the highest temperature was obtained at the focal zone of the ultrasonic transducer lens. As shown in FIG. 1, the test setup included a transducer assembly 10 comprising an ultrasonic transducer having an about 10 cm diameter with an about 10 cm focus. A signal generator 12 was used to control the desired transmission parameters including frequency, amplitude, as well as pulse duration and shape. The signal from the signal generator 12 was amplified by an amplifier 14 which was set up to drive the transducer assembly 10. A computer 16 can direct the signal generator 12.

Mounting and positioning fixtures 18 can be used to hold and position the transducer assembly 10. The mounting and positioning fixtures 18 can include a mounting fixture such as a clamp for holding the transducer assembly 10, and a positioning fixture for moving the transducer assembly 10, for example, a standard manipulation fixture such as a precision scanner. The computer 16 can direct the positioning fixture. Upon receiving a command from the computer 16, the positioning fixture can move the transducer assembly to a desired location and pause for further instructions. Alternatively, upon receiving a command from the computer 16, the positioning fixture can move the transducer assembly in a scanning pattern to cover a target region.

A schematic drawing of a transducer assembly 20 used in these tests is shown in FIG. 2. An ultrasonic transducer 22 consisting of a curved, circular, piezoelectric crystal with an about 10 cm diameter aperture and a curvature radius of about 10 cm producing an about 10 cm focal distance was mounted on an about 20 mm high circular cylinder 24 having an inner diameter of about 9.7 cm and an outer diameter of about 10.2 cm. The crystal (custom ordered from Fuji & Co., 3F, Ichioka Kosan Bldg., 1-4-6 Kawaramachi, Chuo-ku, Osaka, Postal Code 541-0048, Japan) was made of PZT-4, a ceramic material with high piezoelectric performance characteristics. The resonance frequency of the crystal was about 479 KHz. As is well known in the field, the crystal has two electrodes consisting of thin metal deposited on the front and back surface of the crystal, the front surface being the concave surface of the crystal, and the back surface being the convex surface of the crystal. The front concave surface serves the purpose of focusing the ultrasonic waves. One end of a 50 ohm coaxial cable containing a core wire and a ground wire was connected to the crystal, the core wire being connected to the electrode on the front surface of the crystal, and the ground wire being connected to the electrode on the back surface of the crystal. The wires can be connected to the electrodes using methods well known in the art such as soldering and connecting with conductive adhesives. The other end of the coaxial cable was connected to a plug of the BNC type. The transducer assembly 20 was connected to an amplifier by a 50 ohm coaxial cable coming from the amplifier and connecting to the BNC plug attached to the piezoelectric crystal.

The curved piezoelectric crystal can be circular, elliptical or other shapes depending on the desired shape of the confined heated zone.

The cylinder 24 and a bottom cover 26 were machined out of about 5 mm Plexiglas. The bottom cover 26 was bonded to the cylinder 24. Bonding can be performed using an adhesive such as a superglue type of adhesive. Bonding the bottom cover 26 provides an air backing 28 behind the transducer 22.

The piezoelectric crystal was mounted on the cylinder 24 by bonding with an adhesive such as a superglue type of adhesive.

The transmission frequency of the ultrasonic transducer 22 dictates the diameter of the treated zone where about 100 KHz can produce a zone with an estimated focal diameter of about 15 mm, whereas about 1 MHz can produce a focal zone with an estimated diameter of about 1.5 mm. The use of the air backing 28 behind the piezoelectric crystal can provide for effective transmission of acoustic energy in the concave direction of the curved piezoelectric crystal.

Wave energy can be directed into a water medium for coupling the wave to the body of a treated patient. To provide the water medium, a transducer assembly can be inserted into a bag of water, for example a plastic bag, and the bag can be held in contact with the patient's skin. The dimensions of the bag should be sufficient to allow an ultrasonic wave to be focused onto a desired treatment area. The thickness of the bag should be sufficient to provide structural integrity without causing the ultrasonic wave to become ineffective through attenuation. Preferably, the wall of the bag can range in thickness from about 2 microns to about 7 microns. To assure good ultrasonic coupling, a liquid or paste, commonly called a couplant in medical ultrasonics, can be applied to the skin at the point of contact with the bag. Some examples of a couplant are glycerin, grease, burned honey, water, alcohol, and many others well known in the art.

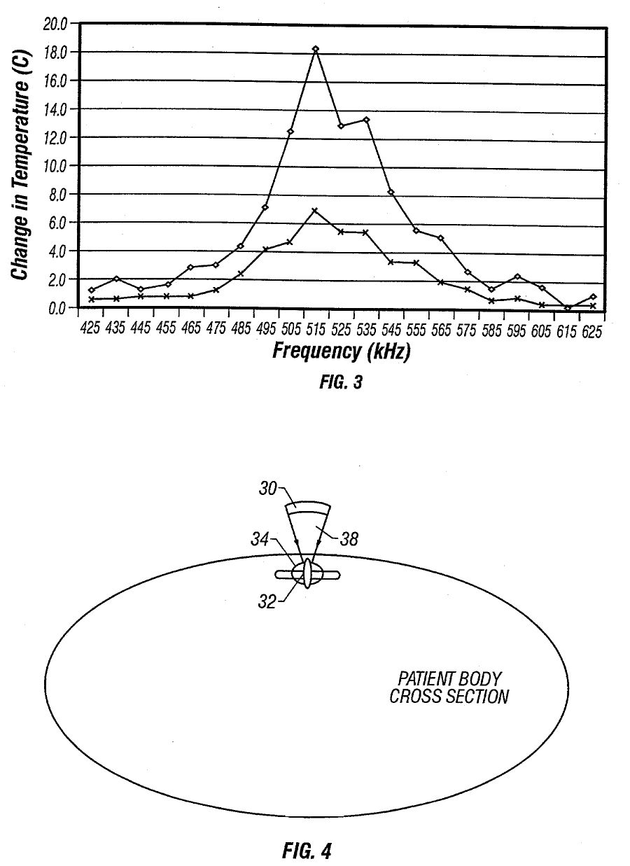

A graph of temperature rise in the focal zone as a function of frequency is shown in FIG. 3, where it is observed that the phenomenon is resonant in nature and can be enhanced by proper selection of the frequency. The upper line shows the temperature rise for a wave focused into water, giving a maximum rise of about 18.5° C. over ambient temperature. The lower line shows the temperature rise for a wave focused into propanol, giving a maximum rise of about 7° C. over ambient temperature. To measure temperature rise in water, a transducer assembly was immersed in a tank filled with water, and a thermocouple was inserted into the water at the center of the focal zone. To measure temperature rise in propanol, a transducer assembly and a sealed plastic bag filled with propanol were immersed in a water-filled tank. A thermocouple was inserted into the sealed bag to measure temperature at the focal zone. However, non-invasive mapping of the temperature distribution in 3D, using magnetic resonance imaging, can also be used. Temperatures as high as about 125° C. were recorded using a continuous wave at the level of about 450-mVolts with about 50-dB amplification. When using a continuous wave, air bubbles were produced in the wave path and a standing wave was formed; both interfere with the traveling wave. For an effective treatment, pulses eliminated these effects and also minimized the potential of overheating.

Thermal Modification of Muscle Tissue by Focused Ultrasonic Waves

In a specific implementation, bovine skeletal muscle samples were subjected to heating by focused ultrasonic waves and test results were highly successful in thermally modifying the area receiving the exposure. In this initial series, tests were conducted for a total duration of about five minutes while experimental parameters were varied. For example, the exposure location was varied to be either internal to the muscle or onto its surface. Also, the excitation power was varied from about 1 to about 50 Watts. Further, the characteristics of the wave, either continuous or pulsed signal, were varied. In the experiments, the temperature level was raised up to about 72° C., which is sufficiently high to lead to destruction of the tissues that were exposed to the treatment.

Gross discoloration of the tissues was evident for the surface experiments. In cases where the focus region was directed internally, histological processing of the tissues was used to assess the effect of the thermal insult. In such cases, a primary damage zone was observed where the muscle fibers were consistently removed. Additionally, surrounding the primary damage zone was another zone of secondary damage, which decreased in intensity with a transition to completely normal tissue. The size of the primary damage zone was about 5-mm, while the extent of the secondary zone extended about 7-mm radially from the primary zone.

The level of ultrasonic energy needs to be controlled to assure the induced thermal exposure is safe and effective in producing the desired cross-linking of the collagen in the ligamentous tissues. The excitation power can be selected from about 1 Watt up to about 50 Watts to allow obliterating the core of the intervertebral disk structure (nucleus pulposus) with minimal damage to the surrounding tissues. The ultrasonic wave control parameters can include pulse shape such as a square-wave or sine-wave, signal amplitude ranging from about 1 to about 20 Volts prior to entering the amplifier, spectral characteristic ranging from about 100-KHz to about 4 MHz, and pulse characteristics such as duration, settling time and period between pulses, that range from microseconds to seconds. Generally, it is preferable to use a frequency as high as about 700 to about 900 KHz since a narrower focal zone is obtained in the range of about a 2 mm diameter. A smaller focus allows for greater clinical control over the treated area.

Treatment Setup

A schematic view of a treatment setup is shown in FIG. 4. A transducer assembly 30 can be positioned with the focal zone 32 located at the desired treatment area of the spine 34 in a patient's body. A water-coupling bag 38 can provide an effective path for the transition of the wave from the transducer assembly 30 to the treated area, while a signal generator can induce the required signals with controlled duration, amplitude and spectral range.

What is claimed:

1. A method of heating a tissue of the body of a life form, comprising:
(a) directing pulsed ultrasonic waves to a selected site of the tissue; and
(b) depositing at the selected site an amount of ultrasonic wave energy sufficient to heat the selected site without adversely damaging other areas of the body.

2. The method of claim 1 in which the pulsed ultrasonic waves are pulsed focused ultrasonic waves.

3. The method of claim 1 in which the tissue is an internal tissue.

4. The method of claim 1 in which the tissue is a ligamentous tissue.

5. The method of claim 4 in which the ligamentous tissue is associated with an intervertebral disc of the spine or a joint of the body.

6. The method of claim 5 in which the ligamentous tissue associated with an intervertebral disc is the nucleus pulposus.

7. The method of claim 1 in which the selected site is heated so that collagen crosslinking at the site is altered without adversely damaging other areas of the body.

8. The method of claim 1 in which the selected site is heated so that tissue in the selected site shrinks without adversely damaging other areas of the body.

9. A method of treating a ligamentous tissue, comprising:
(a) directing pulsed focused ultrasonic waves to a selected site of the tissue; and
(b) depositing at the selected site an amount of ultrasonic wave energy sufficient to heat the site without adversely damaging other areas of the tissue.

10. The method of claim 9 in which the waves have a frequency of about 100 KHz to about 4 MHz.

11. The method of claim 9 in which the waves have a pulse duration of microseconds to seconds.

12. The method of claim 9 in which wave amplitude of the pulsed focused ultrasonic waves is determined by a signal amplitude of about 1V to about 20V before amplification.

13. The method of claim 9 in which the waves have a pulse rate of about 100 repetitions per second to about 1000 repetitions per second.

14. The method of claim 9 in which the waves have a pulse shape in the form of a sine-wave or a square-wave.

15. The method of claim 9 in which the waves have a frequency of about 100 KHz to about 4 MHz, a pulse duration of microseconds to seconds, a pulse rate of about 100 repetitions per second to about 1000 repetitions per second, a pulse shape in the form of a sine-wave or a square-wave, and wave amplitude of the pulsed focused ultrasonic waves is determined by a signal amplitude of about 1V to about 20V before amplification.

16. An apparatus for heating a tissue of the body of a life form, comprising:
(a) an ultrasonic source for producing pulsed ultrasonic waves;
(b) a lens for focusing said pulsed ultrasonic waves on a selected site of the tissue; and
(c) a controller for controlling wave frequency and transmission parameters of said pulsed ultrasonic waves so that ultrasonic wave energy is deposited at the selected site in an amount sufficient to heat the selected site without adversely damaging other areas of the tissue.

17. The apparatus of claim 16 in which the ultrasonic source is an ultrasonic transducer.

18. The apparatus of claim 16 in which the lens is an ultrasonic lens.

19. The apparatus of claim 16 in which the controller is a signal generator.

20. The apparatus of claim 16 in which the ultrasonic transducer comprises both the ultrasonic source and the lens.

21. The apparatus of claim 16 further comprising a temperature monitor for measuring the temperature of the selected site during heating.

22. An apparatus for treating ligamentous tissue comprising:
(a) a transducer assembly comprising an ultrasound transducer for producing pulsed focused ultrasonic waves, the transducer comprising a curved piezoelectric crystal;
(b) a signal generator for controlling wave frequency and transmission parameters of said pulsed ultrasonic waves so that ultrasonic wave energy is deposited at a selected site of the tissue in an amount sufficient to heat the selected site without adversely damaging other areas of the tissue; and
(c) an amplifier for amplifying the signal from the signal generator; said amplifier being coupled to the output of the signal generator, said amplifier driving the ultrasound transducer.

23. The apparatus of claim 22 further comprising a mounting fixture for holding the transducer assembly, and a positioning fixture for moving the transducer assembly.

24. The apparatus of claim 22 in which the waves have a frequency of about 100 KHz to about 4 MHz.

25. The apparatus of claim 22 in which the waves have a pulse duration of microseconds to seconds.

26. The apparatus of claim 22 in which wave amplitude of the pulsed focused ultrasonic waves is determined by a signal amplitude of about 1V to about 20V before amplification.

27. The apparatus of claim 22 in which the waves have a pulse rate of about 100 repetitions per second to about 1000 repetitions per second.

28. The apparatus of claim 22 in which the waves have a pulse shape in the form of a sine-wave or a square-wave.

29. The apparatus of claim 22 in which the waves have a frequency of about 100 KHz to about 4 MHz, a pulse duration of microseconds to seconds, a pulse rate of about 100 repetitions per second to about 1000 repetitions per second, a pulse shape in the form of a sine-wave or a square-wave, and wave amplitude of the pulsed focused ultrasonic waves is determined by a signal amplitude of about 1V to about 20V before amplification.
